(19) United States
(12) Plant Patent Application Publication
(10) Pub. No.: US 2017/0257993 P1
(43) Pub. Date: Sep. 7, 2017

(54) Grapevine named 'IFG Twenty-three'

(50) ;

(71) Applicant: David Cain, Bakersfield, CA (US)

(72) Inventor: David Cain, Bakersfield, CA (US)

(21) Appl. No.: 14/998,924

(22) Filed: Mar. 7, 2016

(51) Int. Cl. A01H 5/08

(52) U.S. Cl. PLT/206

(57) ABSTRACT

This invention is a new and distinct grapevine variety denominated ‘IFG Twenty-three’. The new grapevine is characterized by producing naturally large seedless reddish black berries having a spherical shape with a mild fruity labrusca flavor somewhat similar to the Kyoho variety but smaller in size and seedless.

Latin name of the genus and species claimed: Vitis interspecific hybrid.

Variety denomination: ‘IFG Twenty-three’.

### BACKGROUND OF THE INVENTION

The new and distinct grapevine described and claimed herein originated from a hand pollinated cross of the 01032-067-202, an unnamed seedling selection from the IFG breeding program, and the Arkansas 2798 (non-patented, non-released interspecific selection) hybridized in May 2006. The abortive seed traces were subsequently embryo cultured and the resulting population of plants was planted in the field in April 2007. The present variety of grapevine was selected as a single plant in October 2008 and was first asexually propagated by hardwood cuttings in December 2008 near Delano, Kern County, Calif. The resulting propagules were planted during April 2009 near Delano, Kern County, Calif. and were found to reproduce true-to-type through at least two generations of asexual reproduction.

### BRIEF SUMMARY OF THE INVENTION

The new grapevine ‘IFG Twenty-three’ is characterized by producing naturally large seedless reddish black berries having a spherical shape with a mild fruity labrusca flavor somewhat similar to the Kyoho variety but smaller in size and seedless. Fruits normally ripen mid to late season about early to mid-September near Delano Calif. Fruits are fairly low in acidity, with medium dense firm texture, having occasionally noticeable seed traces and may be difficult to color in hot climatic conditions. Berries may occasionally split at the stylar end. Attachment of the berry to the pedicel is strong. Vines are productive and can be pruned to short spurs. The new grapevine differs from its maternal parent the 01032-067-202 by having dark red rather than light green berries and by having a medium strong fruity labrusca type flavor as opposed to the neutral flavor of the 01032-067-202. ‘IFG Twenty-three’ can be distinguished from its pollen parent, Arkansas 2798, by its dark reddish black color as opposed to full black color, by having round berries as opposed to obovoid berries and by having a mild labrusca flavor rather than a strong labrusca flavor of the Arkansas 2798.

The interspecific nature of the vine is very evident. Vines are very vigorous with a more horizontal growth habit and large leaves as compared to typical varieties of the Vitis vinifera species.

Grape breeders have used several native American grape species to improve hardiness, disease and insect resistance as well as incorporate aromatic flavors into the vinifera species. Previously introduced interspecific varieties have had very limited commercial success due to small fruit size, large seed traces, slipskin texture or lack of firmness. The new grapevine variety ‘IFG Twenty-three’ is being introduced because of its unique combination of labrusca flavor combined with firm texture, seedlessness, good adhesion of skin and flesh and naturally large berry size and its resemblance to the Kyoho grape.

### BRIEF DESCRIPTION OF THE DRAWING

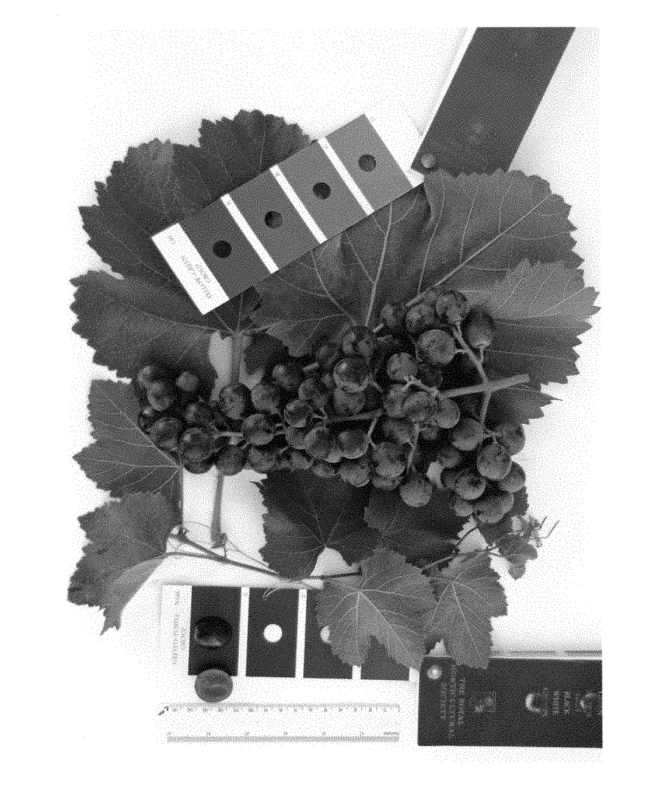

The accompanying photographic drawing in FIG. 1 illustrates in full color ‘IFG Twenty-three’. The photograph was taken outdoors with indirect lighting. The colors are as nearly true as is reasonably possible in a color representation of this type. Mature leaves can be seen on the left side of the drawing. A mature fruit cluster is represented in the center of the drawing. A young shoot tip with tendrils and young leaves can be seen on the right side of the drawing along with a typical berry and a berry in cross-section.

### DETAILED BOTANICAL DESCRIPTION OF THE INVENTION

Throughout this specification, color names beginning with a small letter signify that the name of that color, as used in common speech, is aptly descriptive. Color names beginning with a capital letter designate values based upon R.H.S. Colour Chart, published in 2001 by The Royal Horticultural Society, London, England.

Throughout this specification subjective description values conform to those set forth by the International Plant Genetic Resources Institute publication ‘Descriptors for Grape’ (vitis spp.) (1983) which was developed in collaboration with the Office International de la Vigne et du Vin (OIN) and the International Union for the Protection of New Varieties of Plants (UPOV).

The descriptive matter which follows pertains to ‘IFG Twenty-three’ plants grown in the vicinity of Delano, Kern County, Calif. during 2014, and is believed to apply to plants of the variety grown under similar conditions of soil and climate elsewhere:
Vine:
   General.—Vigor — Vigorous Density of foliage — Dense Productivity — Productive, producing 17.5 to 26.2 kg of fruit per vine Root stock — Own root Training method — Typically spur pruned leaving 2 bud spurs.
   Trunk.—Trunk diameter of 4-year-old vines at 30 cm above the soil line — About 5.8 cm Shape — Somewhat stocky Straps — Very long and continuous Surface texture — Medium rough Inner bark color — The following colors were observed: Greyed-orange: 165A and 200B and 200C.
Shoots:
   Young shoot.—Form of tip — Wide open Distribution of anthocyanin coloration of tip — Piping (striped) Intensity of anthocyanin coloration of tip — Weak Density of prostrate hairs of tip — Dense Density of erect hairs of tip — Absent Color — Yellow-green: 144A.
   Woody shoot (mature canes).—Internode length — Medium, about 15.0 cm Width at node — About 1.7 cm Cross section — Elliptic to narrow elliptic Surface — Striate Main color — The following colors were observed: Greyed-orange: 166A and 166B and 166C and 166D Density of erect hairs on nodes — Medium Density of erect hairs on internodes — Sparse to medium Axillary shoot length at full bloom — Long, approximately 24.9 cm.
   Flowering shoot.—Vigor during flowering — Very strong Attitude during flowering on shoots not tied — Drooping Color — dorsal side of internodes — Yellow-green: 146C, with Red-purple stripes: 59B Color — ventral side of internodes — Yellow-green: 146C Color — dorsal side of nodes — Yellow-green: 146C, with Red-purple stripes: 59B Color — ventral side of nodes — Yellow-green: 146C Density of prostrate hairs on nodes — Sparse Density of erect hairs on nodes — Sparse Density of prostrate hairs on internode — Sparse Density of erect hairs on internode — Very sparse Anthocyanin coloration of buds — Present.
   Tendrils.—Distribution on the shoot (at full flowering) — Discontinuous Length of tendril — Very long, about 39.7 cm Thickness of tendril 2 cm from base — About 3.2 mm Color — Yellow-green: N144A. Tendrils exposed to the sun can also have Red-purple stripes: 186A piped on the dorsal side of the tendrils Form — Bifurcated and trifurcated Number of consecutive tendrils — 2.
Leaves:
   Young leaves.—Color of upper surface of first four distal unfolded leaves — Yellow-green: 144A Average intensity of anthocyanin coloration of six distal leaves prior to flowering — Absent Density of prostrate hairs between veins (lower surface) — Dense Density of prostrate hairs on veins (lower surface) — Sparse Density of erect hairs between veins (lower surface) — Sparse Density of erect hairs on veins (lower surface) — Dense.
   Mature leaves.—Average length — About 14.4 cm Average width — About 20.6 cm Mature leaf size — Large Shape of blade — Wedge-shaped Number of lobes — 5 Blade venation — Palmate Vein color — Yellow-green: 145C Anthocyanin coloration of main veins on upper side of blade — Absent to very weak Mature leaf profile — Undulate Blistering surface of blade upper surface — Weak to medium Leaf blade tip — In the plane of the leaf Undulation of margin — Medium Undulation of blade between main and lateral veins — Overall Shape of teeth — Mixture of both sides straight and both sides convex Length of teeth — Short Ratio length/width of teeth — Medium Shape of upper lateral sinuses — Lobes slightly overlapping Depth of upper lateral sinuses — Shallow General shape petiole sinus — Slightly open to closed Shape of base of upper leaf sinuses — V-shaped Tooth at petiole sinus — Occasionally present Density of prostrate hairs between veins on lower surface of blade — Sparse Density of erect hairs between veins on lower surface of blade — Sparse Density of prostrate hairs on main veins on lower surface of blade — Sparse Density of erect hairs on main veins on lower surface of blade — Dense Density of prostrate hairs on main veins on upper surface of blade — Sparse if Density of erect hairs on main veins on upper surface of blade — Very sparse Autumn coloration of leaves — Leaves can be a single color or a combination of colors, in a mottled pattern or on the edges of the leaves. The following colors were observed: Greyed-purple: 187A and 187B, and N186A and N186B and N186C and N186D, Greyed-orange: 34A and 34B, and Purple: N77A.
   Upper surface.—Color — Yellow-green: 146A Anthocyanin coloration of main veins — Absent to very weak Surface appearance — Semi-glossy Blistering surface of blade — Weak to medium.
   Lower surface.—Color — Yellow-green: 147B Anthocyanin coloration of main veins (lower surface) — Weak Glossiness — Weak Surface texture — Rugose Surface appearance — Dull.
   Petiole.—Length — About 11.2 cm Diameter of petiole 2 cm from blade — About 4.0 mm Petiole color — The following colors were observed: Yellow-green: 145B and 145C and Red-purple: 59A Length of petiole compared to middle vein — Slightly shorter Density of prostrate hairs on petiole — Sparse Density of erect hairs on petiole — Dense.
   Buds.—Bud fruitfulness — Basal: mostly fruitful Position of first fruitful shoot on previous season cane — 1 ^{st} to 2^{nd} node Dormant bud length — About 8.1 mm Dormant bud width in the proximal/distal plane — About 4 7 mm Dormant bud color — Greyed-orange: 166A Time of bud burst — Midseason; Mar. 8, 2014.
Flowers:
   General.—Flower sex — Hermaphrodite Length of first inflorescence — Long, about 18.4 cm long by 8.6 cm wide Position of first flowering and fruiting node — 3^{rd node} (current season growth) Number of inflorescence per flowering shoot — 2 to 3, about 2.1 Time of bloom — Early to medium as compared with similar varieties in the growing area of Delano, Calif. Date of full bloom — Apr. 25, 2014.
Fruit:
   General.—Ripening period — Late: approximately Sep. 16, 2014 Use — Fresh market Keeping quality — Medium, remains commercially acceptable when stored up to 4 weeks at 0° C. and high relative humidity Resistance to — Insects: Average typical of Vitis vinifera species. Diseases: Average typical of Vitis vinifera species. Refractometer test — Soluble solids: about 18.6 Brix Brix/acid — About 56.4 Titratable acidity — About 0.33 Juice pH — About 4.02.
   Cluster.—Mature cluster length (peduncle excluded) — About 26.6 cm Mature cluster width — About 13.9 cm Mature cluster weight — About 583 g Bunch density — Medium loose Number of berries — About 113 Form — Conical.
   Peduncle.—Lignification of peduncle — Weak Length of peduncle — Long, approximately 7.9 cm.
   Berry.—Uniformity of size — Uniform Single berry weight — About 6.3 g natural to about 7.3 g when treated with gibberellic acid Shape — Round Seeds — Contains small rudimentary seed traces, seed traces occasionally noticeable Cross-section — Circular Berry dimensions — Longitudinal axis: about 2.3 cm. Horizontal axis: about 2.1 cm Berry firmness — Firm Particular flavor — Labrusca Bloom (cuticular wax) — Medium heavy Berry separation from pedicel — Medium strong Flesh color — The following colors were observed: Greyed-white: 156B and 156C Skin color (without bloom) — The following colors were observed: Greyed-purple: N186A and N186B and N186C.
   Skin.—Thickness — Medium Skin toughness — Medium to thin; not objectionable when chewing, may occasionally split at the stylar end Reticulation — Absent Tenacity — Tenacious to flesh.

What is claimed:

1. A new and distinct variety of grapevine as herein illustrated and described.
